(19) United States
(12) Patent Application Publication
(10) Pub. No.: US 2018/0275116 A1
(43) Pub. Date: Sep. 27, 2018

(54) GAS SENSOR

(71) Applicants: NGK SPARK PLUG CO., LTD., Nagoya-shi (JP); SPIROSURE, INC., Pleasanton, CA (US)

(72) Inventors: Takayuki KUMAZAKI, Inuyama-shi (JP); Masatoshi UEKI, Niwa-gun (JP); Takafumi SHICHIDA, Kasugai-shi (JP); Kenji NISHIO, Komaki-shi (JP); Tsuyoshi INOUE, Ama-gun (JP); Shigeya AOYAMA, Komaki-shi (JP); Solomon SSENYANGE, Fremont, CA (US); Ryan LEARD, Oakland, CA (US)

(73) Assignees: NGK SPARK PLUG CO., LTD., Nagoya-shi (JP); SPIROSURE, INC., Pleasanton, CA (US)

(21) Appl. No.: 15/467,301

(22) Filed: Mar. 23, 2017

(51) Int. Cl. G01N 33/497; G01N 27/406; G01N 27/407; G01N 33/00

(52) U.S. Cl. CPC G01N 33/497; G01N 27/4067; G01N 2033/4975; G01N 33/0037; G01N 27/407

(57) ABSTRACT

A gas sensor including an adjustment unit having a conversion element for converting a gas component contained in exhaled breath introduced into a first chamber to a particular component, a sensor unit having a second chamber and including a detection element, a ceramic wiring board electrically connected to the detection element, and a single heater for heating the conversion element and the detection element. The ceramic wiring board has an opening, and a ceramic thin plate is stacked on the ceramic wiring board to cover the opening. The ceramic thin plate partially constitutes the adjustment unit and the sensor unit and separates the first chamber and the second chamber from each other. The adjustment unit, the sensor unit, and the heater are integrated in such a manner that the adjustment unit and the sensor unit are thermally coupled through the ceramic thin plate.

### BACKGROUND OF THE INVENTION

### Field of the Invention

The present invention relates to a gas sensor for detecting the concentration of a gas component such as nitrogen oxide (NOx) contained in exhaled breath.

### Description of the Related Art

Environmental control, process control, health care, etc., require measurement of the concentration of NOx contained in a gas under measurement. In particular, diagnosis of asthma requires measurement of NOx contained in exhaled breath at a very low concentration (several ppb to several hundred ppb).

In view of these requirements, a technique of converting NO in exhaled breath to NO₂ has been proposed, using a catalyst and detecting the NO₂ with a sensor element (see US Patent Application Publication No. 2015/0250408 incorporated herein by reference in its entirety, including but not limited to, FIGS. 4, 5A, 5B). In this technique, catalyst in the form of a film is provided on a ceramic substrate; a sensor element is fixedly suspended on another ceramic substrate; and these ceramic substrates are stacked together with a plurality of ceramic substrates for forming gas flow passages to complete a sensor. Further, a heater (heat generation resistor) for activating the catalyst and a heater (heat generation resistor) for heating the sensor element are provided so as to heat the catalyst and the sensor element for stable operation.

In the above-described technique, the catalyst and the sensor element are heated by separate heaters, and therefore, the structure of the sensor tends to become complicated. In order to simplify the structure of the sensor for size reduction, a structure can be employed in which the catalyst and the sensor element are heated by a single (common) heater. However, since the ceramic substrate has a low thermal conductivity and the heat generated by the heater dissipates in the surface direction of the ceramic substrate without being sufficiently transmitted to the catalyst and the sensor element, the size of the heater must be increased. This hinders efforts at reducing the size of the sensor, while increasing the power consumption of the heater.

### SUMMARY OF THE INVENTION

It is therefore an object of the present invention to provide a gas sensor which includes a single heater and whose size and power consumption are reduced.

The above object has been achieved, in a first aspect of the invention, by providing (1) a gas sensor which comprises an adjustment unit which has a first chamber into which exhaled breath is introduced, the adjustment unit including a conversion element for converting a gas component contained in the exhaled breath introduced into the first chamber to a particular component; a sensor unit which has a second chamber into which the exhaled breath having passed through the adjustment unit is introduced, the sensor unit including a detection element having an electric characteristic which changes with a change in concentration of the particular component; a ceramic wiring board which is electrically connected to the detection element and which is at least partially accommodated in the sensor unit; and a single heater for heating the conversion element and the detection element. The ceramic wiring board has an opening penetrating the ceramic wiring board in a thickness direction thereof, and a ceramic thin plate thinner than the ceramic wiring board is stacked on a peripheral edge portion of the ceramic wiring board around the opening and covers the opening. The ceramic thin plate constitutes at least a portion of the adjustment unit and at least a portion of the sensor unit and separates the first chamber and the second chamber from each other. The detection element is disposed on one surface of the ceramic thin plate. Further, the adjustment unit, the sensor unit, and the heater are integrated in such a manner that the adjustment unit and the sensor unit are thermally coupled through the ceramic thin plate.

In the gas sensor according to the first aspect (1) of the invention, the adjustment unit and the sensor unit can be heated by a single heater. Therefore, as compared with the case where separate heaters are provided for the two units, the structure of the gas sensor can be simplified, and the size of the gas sensor can be reduced.

Also, the sensor unit and the adjustment unit are thermally coupled through the ceramic thin plate, and the two units and the heater are integrated. Therefore, even though the ceramic thin plate is present between the heater and one or both of the two units, the two units can be reliably heated at a low electric power by the single heater. This is because the heat of the heater easily conducts to the units through the ceramic thin plate which is thinner and which has a lower thermal resistance than the ceramic wiring board around the ceramic thin plate.

Also, because the detection element of the sensor unit is heated to its operation temperature by the heater as described above, the particular component can be detected stably, whereby the detection accuracy of the particular component can be improved.

Further, since a portion of members constituting the adjustment unit and a portion of members constituting the sensor unit are formed by the ceramic thin plate which is a member common between the two units, it becomes possible to reduce the number of the components of the gas sensor and to reduce the size of the gas sensor.

The above object has also been achieved, in accordance with a second aspect of the invention, by providing (2) a gas sensor which comprises an adjustment unit which has a first chamber into which exhaled breath is introduced, the adjustment unit including a conversion element for converting a gas component contained in the exhaled breath introduced into the first chamber to a particular component; a sensor unit which has a second chamber into which the exhaled breath having passed through the adjustment unit is introduced, the sensor unit including a detection element whose having an electric characteristic which changes with a change in concentration of the particular component; a ceramic wiring board which is electrically connected to the detection element and which is at least partially accommodated in the sensor unit; and a single heater for heating the conversion element and the detection element. The ceramic wiring board has an opening penetrating the ceramic wiring board in a thickness direction thereof. A ceramic flange plate thinner than the ceramic wiring board is formed integrally with the detection element to extend outward from the detection element, and the flange plate is stacked on a peripheral edge portion of the ceramic wiring board around the opening and covers the opening. The flange plate constitutes at least a portion of the adjustment unit and at least a portion of the sensor unit and separates the first chamber and the second chamber from each other. Further, the adjustment unit, the sensor unit, and the heater are integrated in such a manner that the adjustment unit and the sensor unit are thermally coupled through the flange plate.

In the gas sensor according to the second aspect of the present invention, the adjustment unit and the sensor unit can be heated by the single heater. Therefore, as compared with the case where separate heaters are provided for the two units, the structure of the gas sensor can be simplified, and the size of the gas sensor can be reduced.

Also, the sensor unit and the adjustment unit are thermally coupled through the flange plate, and the two units and the heater are integrated. Therefore, even though the flange plate is present between the heater and one or both of the two units, the two units can be reliably heated at a low electric power by the single heater. This is because the heat of the heater easily conducts to the units through the flange plate which is thinner and which has a lower thermal resistance than the ceramic wiring board around the flange plate.

Also, because the detection element of the sensor unit is heated to its operation temperature by the heater as described above, the particular component can be detected stably, whereby the detection accuracy of the particular component can be improved.

Further, since a portion of members constituting the adjustment unit and a portion of members constituting the sensor unit are formed by the flange plate which is a member common between the two units, it becomes possible to reduce the number of the components of the gas sensor and to reduce the size of the gas sensor.

Further, since the detection element and the flange plate are integrated, it is unnecessary to subsequently stack the detection sensor. Therefore, the number of components can be reduced, and the production efficiency can be improved.

In a preferred embodiment (3), the gas sensor of the first aspect (1) further comprises a bonding layer having a lower density than the ceramic wiring board and the ceramic thin plate, the bonding layer being interposed between the ceramic wiring board and the ceramic thin plate.

In the gas sensor (3), the bonding layer, which has a lower density than the ceramic wiring board and the ceramic thin plate, has a higher thermal resistance than the ceramic wiring board and the ceramic thin plate. Therefore, the bonding layer having a high thermal resistance can prevent the escape of heat from the ceramic thin plate to the ceramic wiring board. As a result, heat can be effectively transmitted in the thickness direction of the ceramic thin plate whose thermal resistance is low, whereby the power consumption of the heater is further decreased.

In a preferred embodiment (4), the gas sensor of the second aspect (2) further comprises a bonding layer having a lower density than the ceramic wiring board and the flange plate, the bonding layer being interposed between the ceramic wiring board and the flange plate.

In the gas sensor (4), the bonding layer, which has a lower density than the ceramic wiring board and the flange plate, has a higher thermal resistance than the ceramic wiring board and the flange plate. Therefore, the bonding layer having a high thermal resistance can prevent the escape of heat from the flange plate to the ceramic wiring board. As a result, heat can be effectively transmitted in the thickness direction of the flange plate whose thermal resistance is low, whereby the power consumption of the heater is further decreased.

The present invention can reduce the size and power consumption of a gas sensor having a single heater.

### BRIEF DESCRIPTION OF THE DRAWINGS

FIG. 1 is an exploded perspective view of a gas sensor 1A according to an embodiment of the first aspect of the present invention;

FIG. 2 is a cross-sectional view taken along line A-A of FIG. 1;

FIG. 3 is an exploded perspective view of a ceramic wiring board and a detection element in the gas sensor 1A according to the embodiment of the first aspect of the present invention;

FIG. 4 is a cross-sectional view of a gas sensor 1B according to an embodiment of the second aspect of the present invention, taken along the stacking direction thereof; and

FIG. 5 is an exploded perspective view of a ceramic wiring board and a detection element in the gas sensor 1B according to the embodiment of the second aspect of the present invention.

### DESCRIPTION OF THE PREFERRED EMBODIMENTS

The present invention will now be described in detail with reference to the drawings. However, the present invention should not be construed as being limited thereto.

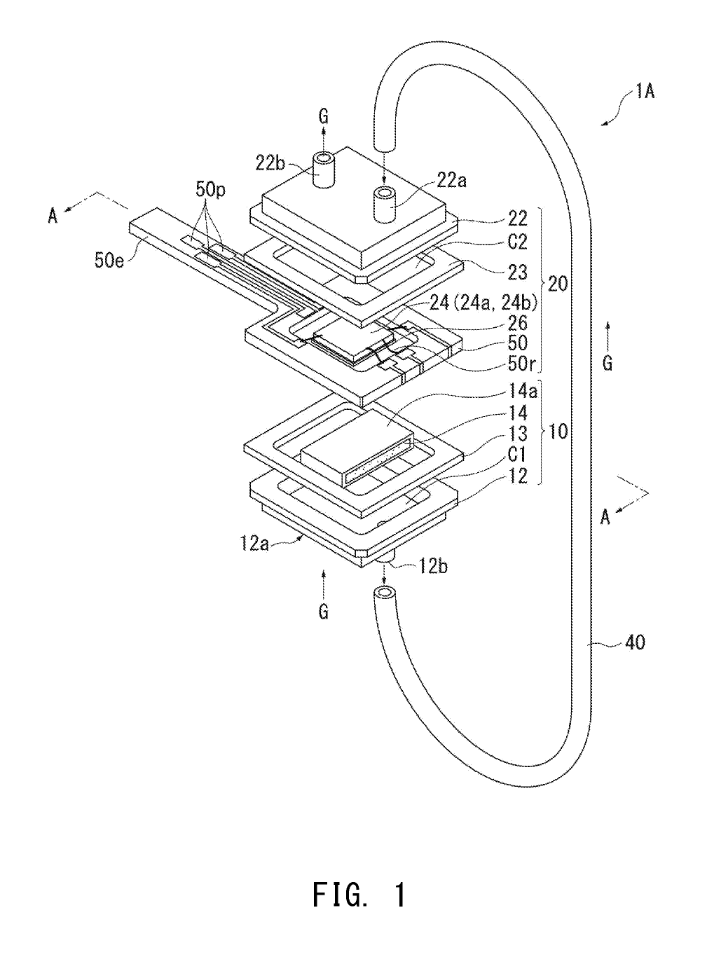

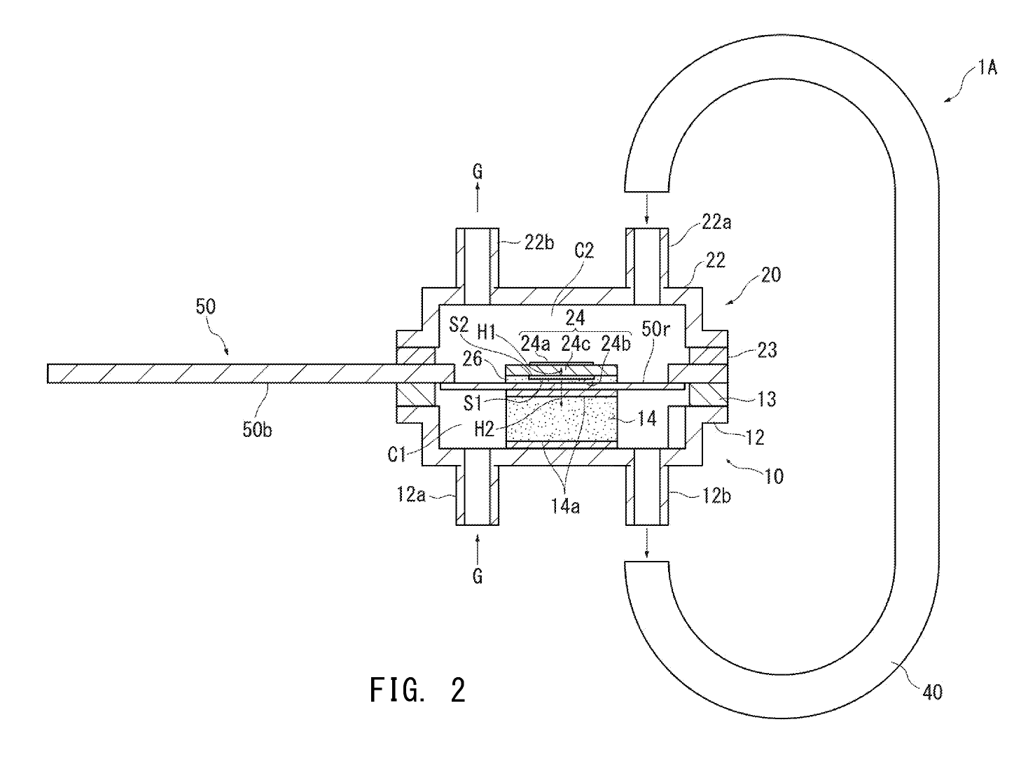

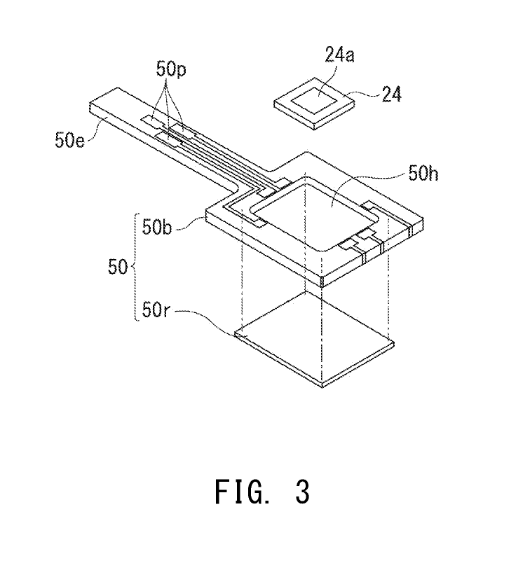

First, a gas sensor 1A according to an embodiment of the first aspect of the present invention will be described with reference to FIGS. 1 to 3. FIG. 1 is an exploded perspective view of the gas sensor 1A according to the embodiment of the first aspect of the present invention. FIG. 2 is a cross-sectional view taken along line A-A of FIG. 1. FIG. 3 is an exploded perspective view of a ceramic wiring board 50 and a detection element 24 in the gas sensor 1A.

As shown in FIG. 1, the gas sensor 1A includes an adjustment unit 10, a sensor unit 20, a gas flow pipe 40, and a plate-shaped ceramic wiring board 50, and has a box-like shape as a whole.

The adjustment unit 10 includes a generally rectangular box-shaped casing 12 which is formed of a metal, has a flange and has an opening in its upper surface (surface facing toward the upper side of FIG. 1); a rectangular frame-shaped packing 13 which is bonded to the flange of the casing 12 via an adhesive layer (not shown); and a conversion element 14 accommodated in the casing 12. The flange of the casing 12 and a peripheral portion of the lower surface of the ceramic wiring board 50 are fixed to the packing 13 via respective adhesive layers (not shown). Thus, the ceramic wiring board 50 closes the opening of the casing 12, and the interior space of the casing 12 serves as a first chamber C1.

Pipe-shaped inlet 12a and outlet 12b which serve as pipe connection ports protrude from the lower surface of the casing 12 such that the inlet 12a and the outlet 12b are separated from each other. The inlet 12a and the outlet 12b communicate with the first chamber C1.

The conversion element 14 is disposed in the first chamber C1 so as to be located between the inlet 12a and the outlet 12b and has the shape of a rectangular parallelepiped. The conversion element 14 is porous and is gas permeable. A seal material 14a made of inorganic fibers (e.g., alumina fibers) is provided on surfaces of the conversion element 14 so as to seal the gap between the surfaces of the conversion element 14 and corresponding wall surfaces of the first chamber C1 (and the lower surface of the ceramic wiring board 50).

Exhaled breath G introduced into the first chamber C1 through the inlet 12a comes into contact with the conversion element 14, and a gas component contained in the exhaled breath G is converted to a particular component. The exhaled breath G is discharged to the outside of the adjustment unit 10 through the outlet 12b. The conversion element 14 contains a catalyst, such as platinum-bearing zeolite, which converts the gas component (specifically, NO) contained in the exhaled breath G to the particular component (specifically, NO₂).

The sensor unit 20 includes a casing 22 having a shape identical with or similar to that of the casing 12, made of a metal, and having an opening in its lower surface; a rectangular frame-shaped packing 23 stacked on the flange of the casing 22; a sensor element unit 24 disposed in the casing 22; a bonding layer 26 for bonding the sensor element unit 24 to a predetermined position of the ceramic wiring board 50 (specifically, a ceramic thin plate 50r described below); and the above-mentioned ceramic wiring board 50. The flange of the casing 22 and a peripheral portion of the upper surface of the ceramic wiring board 50 are fixed to the packing 23 via respective adhesive layers (not shown). Thus, the ceramic wiring board 50 closes the opening of the casing 22, and the interior space of the casing 22 serves as a second chamber C2.

The sensor element unit 24 has a generally rectangular plate-like shape. As shown in FIG. 2, the sensor element unit 24 includes a base portion 24c, a detection element 24a disposed on the upper surface (surface facing toward the upper side of FIG. 1) of the base portion 24c, and a heater 24b disposed on the lower surface of the base portion 24c. Namely, the sensor element unit 24 has an integral structure in which the detection element 24a and the heater 24b are stacked on the upper and lower surfaces, respectively, of the base portion 24c.

As shown in FIG. 3, the ceramic wiring board 50 has a main plate 50b and a ceramic thin plate 50r formed to have a thickness smaller than that of the main plate 50b. The main plate 50b has a generally rectangular frame-shaped portion and a narrow strip-shaped neck portion extending outward from one side of the frame-shaped portion to thereby form an end portion 50e. The frame-shaped portion of the main plate 50b has an opening 50h at its center. The ceramic thin plate 50r, which is larger in size than the opening 50h, is stacked, from the lower side, onto the lower surface of the frame-shaped portion of the main plate 50b surrounding the opening 50h, to thereby cover the opening 50h.

Notably, the main plate 50b and the ceramic thin plate 50r may be formed by stacking un-fired green sheets for the main plate 50b and the ceramic thin plate 50r and firing the stacked green sheets. In this case, the main plate 50b and the ceramic thin plate 50r can be bonded together without use of adhesive or the like. However, the main plate 50b and the ceramic thin plate 50r may be bonded together through use of adhesive as described below. Also, in the present embodiment, the ceramic material used to form the main plate 50b and the ceramic material used to form the ceramic thin plate 50r are the same material (for example, both the ceramic materials contain alumina as a main component).

The sensor element unit 24 (and its detection element 24a) is fixed to the upper surface of the ceramic thin plate 50r such that the heater 24b comes into contact with the upper surface of the ceramic thin plate 50r via the bonding layer 26.

Referring back to FIG. 2, pipe-shaped inlet 22a and outlet 22b which serve as pipe connection ports protrude from the upper surface of the casing 22 such that the inlet 22a and the outlet 22b are separated from each other. The inlet 22a and the outlet 22b communicate with the second chamber C2.

In the second chamber C2, the sensor element unit 24 is disposed on the ceramic thin plate 50r to be located between the inlet 22a and the outlet 22b. The inlet 22a is connected to the outlet 12b through the gas flow pipe 40. The exhaled breath G which has passed through the adjustment unit 10 and whose gas component has been converted to the particular component flows through the gas flow pipe 40 and is introduced into the second chamber C2 through the inlet 22a. As a result, the exhaled breath G comes into contact with the detection element 24a, whereby the concentration of the particular component is measured. The exhaled breath G is then discharged to the outside of the sensor unit 20 through the outlet 22b.

The detection element 24a has an electrical characteristic which changes with the concentration of the particular component. The concentration of the particular component is detected by detecting the changed electrical characteristic. The heater 24b heats the detection element 24a to an operation temperature when energized. The output terminals of the detection element 24a and the energization terminals of the heater 24b are electrically connected to the ceramic wiring board 50 through unillustrated bonding wires.

The base portion 24c can be formed through use of, for example, an insulating ceramic substrate. The detection element 24a may be an NOx sensor element which is composed of a known mixed-potential-type sensor having a solid electrolyte body and a pair of electrodes. The heater 24b may be, for example, a heat generation resistor composed of a meandering conductor formed on the surface of the base portion 24c.

As described above, the end portion 50e (on the left side of FIG. 1) of the ceramic wiring board 50 is rendered narrower than the casings 12 and 22 and extends to the outside of the casings 12 and 22 (the left side of FIG. 1). A plurality of electrode pads 50p are disposed on the surface (the upper surface side in FIGS. 1 and 3) of the end portion 50e. The electrode pads 50p are electrically connected to the detection element 24a and the heater 24b through the above-described bonding wires and wiring (lead conductors) formed on the surface of the ceramic wiring board 50. An electric signal output from the detection element 24a is output to the outside through the electrode pads 50p of the ceramic wiring board 50, and electric power is externally supplied to the heater 24b through the electrode pads 50p so that the heater 24b generates heat.

As shown in FIG. 2, the ceramic wiring board 50 including the ceramic thin plate 50r constitutes the adjustment unit 10 and the sensor unit 20 and separates the first chamber C1 and the second chamber C2 from each other.

The sensor unit 20 and the heater 24b are thermally coupled by virtue of the heater 24b and the detection element 24a within the sensor unit 20 being stacked together for integration through the base portion 24c. Also, the adjustment unit 10 and the sensor unit 20 are thermally coupled by virtue of the adjustment unit 10 and the sensor unit 20 being stacked together for integration through the ceramic thin plate 50r.

The expression “thermally coupled” means a state in which the adjustment unit 10 and the sensor unit 20 are coupled with the ceramic thin plate 50r without air (with no gap) therebetween.

By virtue of the above-described structure, the adjustment unit 10 and the sensor unit 20 can be heated by the single heater 24b. Therefore, as compared with the case where separate heaters are provided for the two units, the size and power consumption of the gas sensor 1A can be reduced.

Also, since the sensor unit 20 and the heater 24b are integrated, as indicated by an arrow H1 of FIG. 2, the heat of the heater 24b disposed inside the sensor unit 20 easily flows to the detection element 24a without passing through the ceramic thin plate 50r.

Further, since the sensor unit 20 and the adjustment unit 10 are thermally coupled through the ceramic thin plate 50r, as indicated by an arrow H2 of FIG. 2, the heat of the heater 24b easily flows to the adjustment unit 10 (the conversion element 14) through the ceramic thin plate 50r which is thinner and has a lower thermal resistance than the ceramic wiring board 50 therearound. As a result, it is possible to reliably heat the two units 10 and 20 at a low electric power by using the single heater 24b.

Also, since the detection element 24a of the sensor unit 20 is heated to its operation temperature by the heater 24b, the particular component can be detected stably, whereby the accuracy in detecting the particular component can be improved.

Notably, as shown in FIG. 2, in the embodiment of the first aspect, the heater 24b has a plate-like shape, has a lower surface (first surface) S1 and an upper surface (second surface) S2 opposing each other, the conversion element 14 is disposed on the lower surface S1 side, and the detection element 24a is disposed on the upper surface S2 side.

Since the conversion element 14 and the detection element 24a are disposed on opposite sides of the heater 24b, the heat of the heater 24b can be transferred to the conversion element 14 and the detection element 24a without wasting heat. Thus, power consumption can be further reduced.

Also, a portion of members constituting the first chamber C1 of the adjustment unit 10 and a portion of members constituting the second chamber C2 of the sensor unit 20 are formed by the ceramic thin plate 50r which is a member common between the two units.

As a result, through use of the ceramic thin plate 50r which is a member common between the two units, it becomes possible to reduce the number of components of the gas sensor 1A and to reduce the size of the gas sensor 1A.

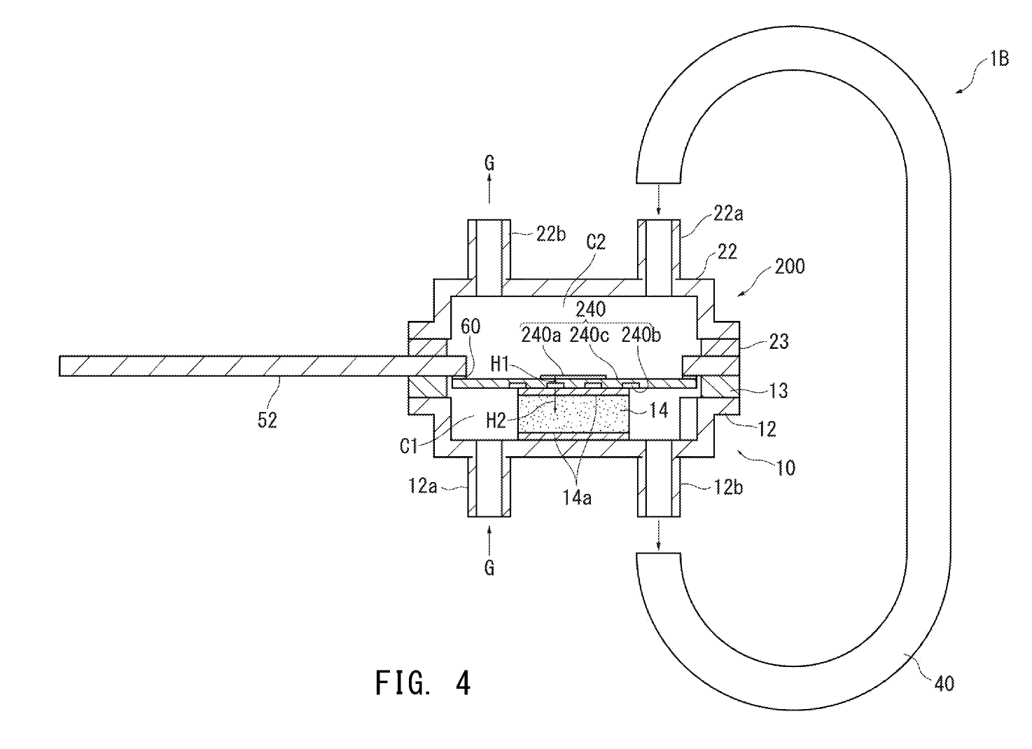

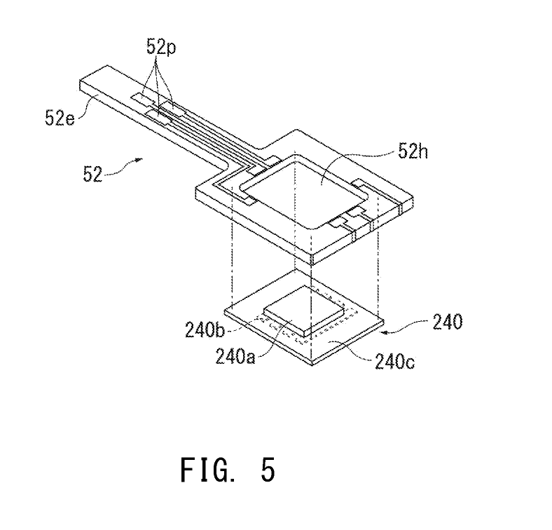

Next, a gas sensor 1B according to an embodiment of the second aspect of the present invention will be described with reference to FIGS. 4 and 5. FIG. 4 is a cross-sectional view of the gas sensor 1B taken along the stacking direction thereof (corresponding to the direction along line A-A of FIG. 1). FIG. 5 is an exploded perspective view of a ceramic wiring board 52 and a detection element 240a in the gas sensor 1B.

As shown in FIG. 4, the gas sensor 1B includes an adjustment unit 10, a sensor unit 200, a gas flow pipe 40, and a plate-shaped ceramic wiring board 52, and has a box-like shape as a whole.

Since the adjustment unit 10 is identical with the adjustment unit 10 in the embodiment of the first aspect, its description will not be repeated.

The sensor unit 200 is identical with the sensor unit 20 in the embodiment of the first aspect, except that the structure of the sensor element unit 240 differs from that in the embodiment of the first aspect and the ceramic thin plate 50r is not used. Portions identical with those in the embodiment of the first aspect are denoted by the same reference numerals, and their descriptions will not be repeated.

The ceramic wiring board 52 is identical with the main plate 50b of the ceramic wiring board 50 in the embodiment of the first aspect. Specifically, the ceramic wiring board 52 has a generally rectangular frame-shaped portion and a narrow strip-shaped neck portion extending outward from one side of the frame-shaped portion to thereby form an end portion 52e. An opening 52h is provided at the center of the frame-shaped portion.

As shown in FIG. 5, the sensor element unit 240 has an integral structure which includes a detection element 240a similar to the detection element in the embodiment of the first aspect, a flange plate 240c made of ceramic, and a heater 240b exposed from the surface (lower surface) of the flange plate 240c opposite the detection element 240a. The flange plate 240c is formed integrally with the detection element 240a to extend outward from the detection element 240a such that the detection element 240a is exposed from the upper surface of the flange plate 240c. The heater 240b for heating the detection element 240a is embedded in the flange plate 240c in such a manner that the lower surface of the heater 240b becomes flush with the lower surface of the flange plate 240c.

The flange plate 240c has a generally rectangular plate-like shape, is larger in size than the opening 52h, and is thinner than the ceramic wiring board 52. Notably, the ceramic material used to form the flange plate 240c and the ceramic material used to form the ceramic wiring board 52 are made of the same material (for example, both the ceramic materials contain alumina as a main component).

The detection element 240a has an electrical characteristic which changes with the concentration of the particular component. The concentration of the particular component is detected by detecting the changed electrical characteristic. The heater 240b heats the detection element 240a to an operation temperature when energized. The output terminals of the detection element 240a and the energization terminals of the heater 240b are electrically connected to the ceramic wiring board 52 through unillustrated bonding wires as in the case of the embodiment of the first aspect. Electrode pads 52p are electrically connected to the above-described bonding wires and wiring (lead conductors) formed on the surface of the ceramic wiring board 52.

An electric signal output from the detection element 240a is output to the outside through the electrode pads 52p of the ceramic wiring board 52, and electric power is externally supplied to the heater 240b through the electrode pads 52p so that the heater 240b generates heat.

The detection element 240a may be an NOx sensor element which is composed of a known mixed-potential-type sensor having a solid electrolyte body and a pair of electrodes. The heater 240b may be, for example, a heat generation resistor composed of a meandering conductor formed on the surface of the flange plate 240c.

The flange plate 240c of the sensor element unit 240 is stacked, from the lower side, onto the lower surface of the frame-shaped portion of the ceramic wiring board 52 surrounding the opening 52h, to thereby cover the opening 52h.

Notably, in the embodiment of the second aspect, the ceramic wiring board 52 and the flange plate 240c are bonded together by a bonding layer 60. The bonding layer 60 is formed by firing an adhesive made of, for example, paste containing ceramic powder. This adhesive is applied between the ceramic wiring board 52 and the flange plate 240c, the ceramic wiring board 52 and the flange plate 240c are pressed together, and the ceramic wiring board 52 and the flange plate 240c are then fired, whereby the ceramic wiring board 52 and the flange plate 240c are bonded together.

The adhesive which is to become the bonding layer 60 is prepared to include a dispersant and an auxiliary which volatilizes as a result of firing so that the adhesive becomes highly flowable and can fill the gap between the ceramic wiring board 52 and the flange plate 240c. Therefore, the bonding layer 60 after being fired is more porous and has a lower density than the ceramic wiring board 52 and the flange plate 240c. Notably, the fact that the density of the bonding layer 60 is lower than that of the ceramic wiring board 52 and the flange plate 240c can be confirmed from a sectional photograph.

As shown in FIG. 4, the ceramic wiring board 52, including the flange plate 240c, constitutes the adjustment unit 10 and the sensor unit 200 and separates the first chamber C1 and the second chamber C2 from each other.

The adjustment unit 10 and the heater 240b are thermally coupled by virtue of the heater 240b being stacked on the adjustment unit 10 for direct contact and integration therewith. Also, the adjustment unit 10 and the sensor unit 200 are thermally coupled by virtue of the adjustment unit 10 and the sensor unit 200 being stacked and integrated together, with the flange plate 240c interposed therebetween.

By virtue of the above-described structure, in the embodiment of the second aspect as well, the adjustment unit 10 and the sensor unit 200 can be heated by the single heater 240b. Therefore, as compared with the case where separate heaters are provided for the two units, the size and power consumption of the gas sensor 1B can be reduced.

Also, since the adjustment unit 10 and the heater 240b are in direct contact with each other, as indicated by an arrow H2 of FIG. 4, the heat of the heater 240b easily flows to the adjustment unit 10 (the conversion element 14) without passing through the flange plate 240c.

Further, since the sensor unit 200 and the adjustment unit 10 are thermally coupled through the flange plate 240c, as indicated by an arrow H1 of FIG. 4, the heat of the heater 240b easily flows to the sensor unit 200 (the detection element 240a) through the flange plate 240c which is thinner and has a lower thermal resistance than the ceramic wiring board 52 therearound. As a result, it is possible to reliably heat the two units 10 and 200 at a low electric power by using the single heater 240b.

Also, since the detection element 240a of the sensor unit 200 is heated to its operation temperature by the heater 240b, the particular component can be detected stably, whereby the accuracy in detecting the particular component can be improved.

Notably, in the embodiment of the second aspect of the present invention, because the detection element 240a of the sensor element unit 240 and the flange plate 240c are integrated, it is unnecessary to subsequently stack the sensor element unit 24 on the ceramic thin plate 50r as in the case of the embodiment of the first aspect. Therefore, the number of components can be reduced, and the production efficiency can be improved.

In the present embodiment, the bonding layer 60 which has a lower density than the ceramic wiring board 52 and the flange plate 240c (namely, the bonding layer 60 which is formed to be more porous than the ceramic wiring board 52), is present between the ceramic wiring board 52 and the flange plate 240c. Since the bonding layer 60 is porous and has a large thermal resistance, the bonding layer 60 having the high thermal resistance prevents the escape of heat from the flange plate 240c to the ceramic wiring board 52. As a result, heat can be effectively transmitted in the thickness direction of the flange plate 240c having a low thermal resistance, whereby the power consumption of the heater 240b is further decreased.

Notably, the advantageous effect achieved by the bonding layer 60 can be similarly attained in the embodiment of the first aspect of the present invention in which a ceramic thin plate is stacked on a ceramic wiring board. Notably, the bonding layer 60 used in the embodiments of the first and second aspects may be formed of a resin, glass, or a like material which has excellent heat resistance.

Needless to say, the present invention is not limited to the above-described embodiments, and encompasses various modifications and equivalents which fall within the scope of the present invention.

The shape, etc., of the gas sensor and the shapes, etc. of the adjustment unit and the sensor unit which constitute the gas sensor are not limited to those employed in the above-described embodiments. No limitation is imposed on the types, etc., of the conversion element and the detection element.

No limitation is imposed on the position of the heater. For example, in the embodiment of the second aspect shown in FIG. 4, the heater 240b may be buried in the flange plate 240c. In this case, the heat of the heater 240b is transferred to the adjustment unit 10 through the flange plate 240c and is transferred to the sensor unit 200 through the flange plate 240c. The same is true of the case where the heater is buried in the ceramic thin plate in the embodiment of the first aspect.

Meanwhile, in the case where the heater 240b is exposed from the lower surface of the flange plate 240c as shown in FIG. 4, or the case where the heater 24b of FIG. 2 is disposed between the ceramic thin plate 50r and the adjustment unit 10 instead of being disposed between the ceramic thin plate 50r and the detection element 24a, the adjustment unit is directly heated by the heater.

In the example of FIG. 2, the ceramic wiring board 50 including the ceramic thin plate 50r constitutes the adjustment unit 10 and the sensor unit 20, and separates the first chamber C1 and the second chamber C2 from each other. However, the ceramic thin plate 50r may be made larger than the adjustment unit 10 and the sensor unit 20, and used solely so as to constitute the adjustment unit 10 and the sensor unit 20 and so as to separate the first chamber C1 and the second chamber C2 from each other.

Similarly, the flange plate 240c shown in FIG. 4 may be made larger than the adjustment unit 10 and the sensor unit 200.

In the above-described embodiments, various members; i.e., the casing 12, the packing 23, the ceramic wiring board 50 (52), the packing 13, and the casing 22, are fixed through use of an adhesive. However, the gas sensor 1A (1B) may be assembled without the use of an adhesive. Specifically, other members may be used to externally apply a force (urging force) toward the ceramic wiring board 50 (52) to the casing 12 and the casing 22 to thereby fix these members such that they do not shift position.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

What is claimed is:

1. A gas sensor comprising:
an adjustment unit which has a first chamber into which exhaled breath is introduced, the adjustment unit including a conversion element for converting a gas component contained in the exhaled breath introduced into the first chamber to a particular component;
a sensor unit which has a second chamber into which the exhaled breath having passed through the adjustment unit is introduced, the sensor unit including a detection element having an electric characteristic which changes with a change in concentration of the particular component;
a ceramic wiring board which is electrically connected to the detection element and which is at least partially accommodated in the sensor unit; and
a single heater for heating the conversion element and the detection element, wherein
the ceramic wiring board has an opening penetrating the ceramic wiring board in a thickness direction thereof, and a ceramic thin plate thinner than the ceramic wiring board is stacked on a peripheral edge portion of the ceramic wiring board around the opening and covers the opening;
the ceramic thin plate constitutes at least a portion of the adjustment unit and at least a portion of the sensor unit and separates the first chamber and the second chamber from each other;
the detection element is disposed on one surface of the ceramic thin plate; and
the adjustment unit, the sensor unit, and the heater are integrated in such a manner that the adjustment unit and the sensor unit are thermally coupled through the ceramic thin plate.

2. A gas sensor comprising:
an adjustment unit which has a first chamber into which exhaled breath is introduced, the adjustment unit including a conversion element for converting a gas component contained in the exhaled breath introduced into the first chamber to a particular component;
a sensor unit which has a second chamber into which the exhaled breath having passed through the adjustment unit is introduced, the sensor unit including a detection element having an electric characteristic which changes with a change in concentration of the particular component;
a ceramic wiring board which is electrically connected to the detection element and which is at least partially accommodated in the sensor unit; and
a single heater for heating the conversion element and the detection element, wherein
the ceramic wiring board has an opening penetrating the ceramic wiring board in a thickness direction thereof;
a ceramic flange plate thinner than the ceramic wiring board is formed integrally with the detection element to extend outward from the detection element, and the flange plate is stacked on a peripheral edge portion of the ceramic wiring board around the opening and covers the opening;
the flange plate constitutes at least a portion of the adjustment unit and at least a portion of the sensor unit and separates the first chamber and the second chamber from each other; and
the adjustment unit, the sensor unit, and the heater are integrated in such a manner that the adjustment unit and the sensor unit are thermally coupled through the flange plate.

3. The gas sensor as claimed in claim 1, further comprising a bonding layer having a lower density than the ceramic wiring board and the ceramic thin plate, the bonding layer being interposed between the ceramic wiring board and the ceramic thin plate.

4. The gas sensor as claimed in claim 2, further comprising a bonding layer having a lower density than the ceramic wiring board and the flange plate, the bonding layer being interposed between the ceramic wiring board and the flange plate.
